SEQUENCE LISTING (7 sequences)

SEQ ID NO: 1 - DNA, 2147 bp, Artificial Sequence
  sequence for deletion of E3gp19k
gatatcattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgatttaaatatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtggccttaaacgcctatttaaattttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttgatatc

SEQ ID NO: 2 - DNA, 3184 bp, Artificial Sequence
  Sequence for inserting human IL-21 into the E3gp19k region
gatatcgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagagttcctagaaagattcaaatcacttctccaaaagatgattcatcagcatctgtcctctagaacacacggaagtgaagattcctgaatttaaatatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtggccttaaacgcctatttaaattttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctagatatc

SEQ ID NO: 3 - DNA, 2699 bp, Artificial Sequence
  Sequence for insertion of Y477A mutation, TAYT deletion, and RDG peptide A20 into the fibre region
ctggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacgatatcttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaagcttggaactttagaaatggagatcttactgaaggcaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactaacgcagtacctaacttgagaggagatctacaggtgttggcacagaaggtcgcacgtactccaagtgcatactctatgtcattttcatgggactggtctggccacaactacattaatgaaatatttgccacatcctcttacactttttcatacattgcccaagaataaatttaaatatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtggccttaaacgcctatttaaatagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaagatatctacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaac

SEQ ID NO: 4 - DNA, 60 bp, Artificial Sequence
  Sequence of A20FMDV2 peptide
aacgcagtacctaacttgagaggagatctacaggtgttggcacagaaggtcgcacgtact

SEQ ID NO: 5 - protein, 20 aa, Artificial Sequence
  A20
NAVPNLRGDLQVLAQKVART

SEQ ID NO: 6 - DNA, 18 bp, Artificial Sequence
  E3 sequencing primer
gggttggggttattctct

SEQ ID NO: 7 - DNA, 21 bp, Artificial Sequence
  fibre region sequencing primer
gacagcacaggtgccattaca

(19) United States
(12) Patent Application Publication
(10) Pub. No.: US 2023/0149485 A1
(43) Pub. Date: May 18, 2023

(54) ENGINEERED ONCOLYTIC ADENOVIRUS

(71) Applicant: SHENZHEN HUA YAO KANG MING BIOPHARMACEUTICAL CO., LTD., Shenzhen City, Guangdong (CN)

(72) Inventor: Ming YUAN, Shenzhen City, Guangdong (CN)

(21) Appl. No.: 17/917,522

(22) PCT Filed: Apr. 7, 2021
(86) PCT No.: PCT/IB2021/052898

§ 371 Date: Oct. 6, 2022

Related U.S. Application Data
(60) Provisional application No. 63/006,115, filed on Apr. 7, 2020.

(51) Int. Cl. A61K 35/761; C12N 15/86; C12N 15/113; C07K 14/54; A61K 45/06; A61P 35/00

(52) U.S. Cl. CPC A61K 35/761; C12N 15/86; C12N 15/113; C07K 14/54; A61K 45/06; A61P 35/00; C12N 2710/10343; C12N 2710/10321

(57) ABSTRACT

Provided is a modified virus Ad5 which is capable of expressing a cytokine, and the modified virus Ad5 is capable of expressing an A20. And also provided are a vector and a cell comprising the modified virus Ad5. The modified virus may be used in cancer treatment.

### BACKGROUND OF THE INVENTION

Adenovirus has been engineered as an oncolytic virus to target tumors specifically with minimal toxicity to normal cells. Safety has been demonstrated in clinical trials with various adenoviral mutants in tens of thousands of patients. However, in the past most clinical trials evaluated Adenovirus mutants were designed to target the frequently dysfunctional p53 activity in human tumors. The first clinical application of this type of Adenovirus was d11520 (Onyx-015; AE1B55K and AF3B). H101, a similar Adenovirus mutant, was licensed for anti-cancer therapy in China (Shanghai Sunway Biotech, China). While the tumor selectivity was demonstrated for these mutants, the efficacy was only shown in combination with chemotherapy. It has later been found that essential functions of the deleted E1B55K- and E3B-genes (such as late viral RNA transport and protection against host-immune defense respectively) contributed to the attenuated efficacy of these viruses.

The ability of Adenovirus to evade host immune surveillance is critical to its persistence. Four immune-regulatory proteins encoded by the E3 region of human Ads have been described previously. One of them, gp19K (also E3/19K), binds heavy chains of the major histocompatibility complex (MHC) class I antigens and inhibits their transport to the cell surface. Therefore, E3gp19K is involved in avoiding recognition and elimination of infected cells by the host immune system, cytotoxic T lymphocytes (CTL).

Human adenoviruses (HAdV/Ad), particularly species C type 5 (HAdV-05/Ad5), have been developed as agents for virotherapy. The mechanisms of Ad5 cellular uptake and tropism in vitro have been clearly understood. Cellular virus uptake occurs via binding of the Ad5 fibre protein to coxsackie and adenovirus receptor (CAR). However, CAR is ubiquitously expressed across human tissues, including erythrocytes and on a variety of tumor cells, and loss of CAR expression in tumors has been documented in a number of reports. Therefore, CAR-utilizing vectors based virotherapy may not be ideal for efficient tumor-targeting, evaluation of alternative receptor tropisms needs to be explored.

In the present application, the inventors report on the generation of a novel mutant, Ad5-3del-A20T-IL21, with incorporation of the A20FMDV2 peptide, ablation of CAR binding, expressing IL21, for optimal replication-selectivity, cancer targeting, and immune stimulation. Ad5-3del-A20T-IL21 was highly efficacious and retained all viral functions necessary for propagation in various cancer cells. We expect these findings to direct further optimization of oncolytic adenoviruses for systemic delivery to improve on therapeutic efficacy in patients with cancer.

### SUMMARY OF THE INVENTION

The present application realizes the deletion of a combination of three viral genes, modifications to fiber region, carrying the immuno-regulatory gene IL21.

The first embodiment of the present application includes generation of the recombinant adenovirus using linearized donor cassette, thereby avoiding the troublesome selection of cloning vector to carry the donor cassette.

The second embodiment of the present application includes filling the gap left upon removal of antibiotic resistant gene using poly-linkers of nucleotides (in this case using restriction enzyme SwaI), which renders modification of multi genes feasible.

The third embodiment of the present application includes creation of backbone adenovirus with deletions of three genes, namely E1ACR2, E1B19k, E3gp19K using the techniques described in the first and second embodiments.

The fourth embodiment of the present application includes creation of a recombinant adenovirus armed with human IL-21 gene inserted into E3gp19K.

The fifth embodiment of the present application includes creation of a recombinant adenovirus with mutation of Y477A, deletion of TAYT, insertion of RGD peptide A20FMDV2 into fibre of adenovirus, based on the virus created by the fourth embodiment.

Furthermore, the A20FMDV2 of the fifth embodiment is precise 20 peptide.

In the sixth embodiment of the present application, no extra peptide attached in the virus created by the fifth embodiment.

In the seventh embodiment of the present application, the virus created by any embodiments of the present application is used for treatment of cancers expressing αvβ6 integrin, including but not limited to, pancreatic cancer, head and neck cancer, and ovarian cancer.

In the eighth embodiment of the present application, the virus created by any embodiments of the present application is used for treatment of cancers via intravenous injection.

Furthermore, in any embodiment of the present application, the virus is adenovirus.

The ninth embodiment of the present application provides a combination of PI3K delta inhibitor with intravenous injection of modified virus created in claim 5 for improving the anti-tumor potency of the modified virus.

The ninth embodiment of the present application provides a combination of check point inhibitor with modified virus created by any embodiment of the present application for improving the anti-tumor potency of the modified virus.

Features of the embodiments of the present application:

1. This product realizes the deletion of a combination of three viral genes, modifications to fiber region, carrying the immuno-regulatory gene IL21.

2. Deletion of E1ACR2 gene allows the mutant virus to replicate selectively in tumor cells sparing the normal cells.

The E1ACR2-region is responsible for binding and inactivation of pRb thereby releasing E2F for S-phase induction of cell cycle. However, in proliferating normal cells and in tumor cells with deregulated cell cycle control (mainly pRb and p16 alterations) the function of E1ACR2-region is redundant.

3. Deletion of E1B19k gene.

It was demonstrated that ΔE1B19K-mutants had increased therapeutic index and lower liver toxicity in vivo, while anti-tumor potency was maintained. The anti-apoptotic E1B19K protein promotes viral replication and spread by blocking Bax-Bak oligomerization and mitochondrial pore-formation analogous to the cellular Bcl-2 homologue. In contrast to the E1B55K protein that mainly inhibits p53-dependent pathways, E1B19K inhibits both death receptor and intrinsically induced apoptosis through p53-dependent and p53independent mechanisms. Adenovirus mutant deleted in both the E1B19K-gene and E1ACR2-region with intact E3-region improved efficacy and selectivity both as a single agent and in combination with standard chemotherapeutics.

4. Deletion of E3gp19k.

Adenovirus E3-gp19K is a transmembrane glycoprotein, localized in the endoplasmic reticulum (ER), which forms a complex with major histocompatibility complex (MEW) class I antigens and retains them in the ER, thereby preventing cytolysis by cytotoxic T lymphocytes (CTL). The ER luminal domain of gp19K, residues 1 to 107, is known to be sufficient for binding to class I antigens; the transmembrane and cytoplasmic ER retention domains are located at residues aa 108 to 127 and 128 to 142, respectively.

5. Mutation Y477A and deletion of TAYT in fibre region.

Ad5 mutant, featuring a set of fiber mutations (Y477A and a TAYT deletion), is supposed to abrogate binding to factor IX (FIX) and C4b-binding protein (C4BP). This mutant would display significantly reduced liver transduction and toxicity, low-level cytokine induction after intravenous delivery.

6. A20

The αvβ6 integrin is highly expressed in many solid tumors but not in normal cells. Adenovirus mutants were engineered to express a 20 amino acid peptide A20FMDV2 derived from the foot-and mouth disease virus (FMDV) that selectively binds through an Arg-Gly-Asp (RGD)-domain to αvβ6.

7. The virus carries interleukin 21 (IL-21) gene

Like interleukin 12, interleukin 21 also activates NK and killer T cells. In the process of immune activation, IL-21 plays a role later than IL-12, and the two interleukins synergistically activate immune cells. The therapeutic gene is inserted into the E3gp19k.

In one aspect, the present application provides a modified virus Ad5, wherein the modified virus Ad5 is capable of expressing a cytokine, and the modified virus Ad5 is capable of expressing an A20.

In some embodiments, the cytokine is originated from human.

In some embodiments, the cytokine comprises an interleukin, a tumor necrosis factor, an interferon, a chemokine, a lymphokine and/or a growth factor.

In some embodiments, the cytokine comprises an IL12, an IL2, an IL15 and/or an IL8.

In some embodiments, the cytokine comprises an IL-21.

In some embodiments, a gene encoding the cytokine is incorporated in to the genome of the modified virus Ad5.

In some embodiments, the A20 is originated from a foot-and mouth disease virus (FMDV).

In some embodiments, the gene encoding the A20 has a nucleic acid sequence as set forth in SEQ ID NO. 4.

In some embodiments, a gene encoding the A20 is incorporated into the genome of the modified virus Ad5.

In some embodiments, a gene encoding the A20 is incorporated into the HI-loop of the modified virus Ad5.

In some embodiments, the incorporation uses a method of gene editing and/or gene recombination.

In some embodiments, the modified virus Ad5 has at least one modification in fibre region.

In some embodiments, the modification in fibre region comprises an amino acid substitution Y477A.

In some embodiments, the modification in fibre region comprises a deletion of amino acids TATY at the residues 489-492.

In some embodiments, compared to a wild virus Ad5, the expression and/or activity of an E1ACR2 gene is downregulated in the modified virus Ad5.

In some embodiments, compared to a wild virus Ad5, the expression and/or activity of a E1B19K gene is downregulated in the modified virus Ad5.

In some embodiments, compared to a wild virus Ad5, the expression and/or activity of a E3gp19K gene is downregulated in the modified virus Ad5.

In some embodiments, compared to a wild virus Ad5, the expression and/or activity of the E1ACR2 gene, the E1B19K gene and the E3gp19K gene are downregulated in the modified virus Ad5.

In some embodiments, the downregulation uses a method of gene editing and/or gene recombination.

In some embodiments, the gene editing uses an anti-sense RNA, a siRNA, a shRNA and/or a CRISPR/C as system.

In some embodiments, at least a portion of the genes encoding the E1ACR2 gene, at least a portion of the E1B19K gene and at least a portion of the E3gp19K are deleted.

In some embodiments, the gene encoding a cytokine is incorporated into the site of the E1ACR2 gene, the E1B19K gene or the E3gp19K gene.

In some embodiments, the modified virus Ad5 is capable of expressing a gene and/or a ligand targeting a T cell, a gene and/or a ligand targeting a tumor cell, and/or a therapeutic gene.

In some embodiments, the therapeutic gene is selected from a group consisting of: a gene encoding an immune co-stimulatory pathway activating molecule, a gene encoding a checkpoint inhibitor, a gene encoding a cytotoxic, a gene encoding a tumor suppressor gene, and an anti-angiogenesis gene.

In some embodiments, the immune co-stimulatory pathway activating molecule is selected from a group consisting of: a CD40 ligand (CD40L), a ICOS ligand, a GITR ligand, a 4-1BB ligand, an OX40 ligand, a TL1A, a CD30 ligand, a CD27, and a Flt3 ligand or the variant thereof.

In some embodiments, the checkpoint inhibitor is selected from a group consisting of: a PD-1 inhibitor, a PD-L1 inhibitor and a CTLA-4 inhibitor.

In some embodiments, the tumor suppressor gene comprises HIC1 gene.

In another aspect, the present application provides an isolated nucleic acid molecule, encoding the modified virus Ad5 of the present application.

In another aspect, the present application provides a vector, comprising the modified virus Ad5 of the present application, and/or the isolated nucleic acid molecule of the present application.

In another aspect, the present application provides a cell, comprising the modified virus Ad5 of the present application, the isolated nucleic acid molecule of the present application, and/or the vector of the present application.

In another aspect, the present application provides a pharmaceutical composition, wherein the pharmaceutical composition comprises the modified virus Ad5 of the present application and a pharmaceutically accepted adjuvant.

In another aspect, the present application provides a method of treating a disease and/or a disorder, comprising administrating the modified virus Ad5 of the present application, the isolated nucleic acid molecule of the present application, the vector of the present application, the cell of the present application, and/or the pharmaceutical composition of the present application to a subject in need of.

In some embodiments, the method comprises administrating the modified virus Ad5 of the present application to a subject in need of in a combination of at least one agent, and the agent is selected from a group consisting of an anti-cancer agent, an agonist, an antagonist, a chemotherapeutic agent and a radiation agent.

In some embodiments, the disease comprises a tumor.

In some embodiments, the disease comprises a tumor expressing αvβ6 integrin.

In some embodiments, the disease comprises a pancreatic cancer, a head and neck cancer, and/or an ovarian cancer.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 shows a schematic diagram of the product.

FIG. 2A-2C show the shuttle cassette for modifications of Ad5. A: The shuttle cassette for the deletion of E3gp19k. The left arm targets the left side of the E3gp19k gene, and the right arm targets the right side of the E3gp19k. The Chloramphenicol and its promoter are located between the left arm and the right arm. The shuttle cassette is cloned into EcoRV sites of pUC57 vector. B: The shuttle cassette for the deletion of E3gp19k. The left arm targets the left side of the E3gp19k gene, and the right arm targets the right side of the E3gp19k. Human IL-21(hIL-21) uses the promoter of E3gp19k, together with the Chloramphenicol and its promoter are located between the left arm and right arm. The shuttle cassette is cloned into EcoRV sites of pUC57 vector. C: The shuttle cassette for mutation Y477A, deletion of TAYT, insertion of A20. The shuttle cassette is cloned into EcoRV sites of pUC57 vector.

FIG. 3 shows the diagram of deletion of E3gp19k. 1) E3gp19k was deleted by combination of shuttle cassette and backbone virus genome in the vector through homologous recombination. The resulted recombinant was selected and grown under Chloramphenicol on LB plate, colonies were picked up, grown and plasmid was extracted, then subject to sequencing to confirm the deletion of E3gp19k. 2) Chloramphenicol was cut out using SwaI from the confirmed recombinant plasmid with E3gp19k deletion and 3) re-ligated to obtain the desired recombinant plasmid, which will be used to generate the modified adenovirus.

FIG. 4 shows the diagram of hIL-21 replacing E3gp19k. 1) E3gp19k gene was replaced by hIL-21 by combination of shuttle cassette and backbone virus genome in the vector through homologous recombination. The resulted recombinant was selected and grown under Chloramphenicol on LB plate, colonies were picked up, grown and plasmid was extracted, then subject to sequencing to confirm the deletion of E3gp19k and. 2) Chloramphenicol was cut out using SwaI from the confirmed recombinant plasmid with insertion of hIL-21 replacing E3gp19k and 3) re-ligated to obtain the desired recombinant plasmid, which will be used to generate the modified adenovirus.

FIG. 5 shows the diagram of generation of mutation of Y477A, deletion of TAYT and insertion of A20 in adenovirus with three deleted genes. 1) Mutation of Y477A, deletion of TAYT and insertion of A20 were achieved by combination of shuttle cassette and backbone virus genome in the vector through homologous recombination. The resulted recombinant was selected and grown under Chloramphenicol on LB plate, colonies were picked up, grown and plasmid was extracted, then subject to sequencing to confirm the mutation of Y477A, deletion of TAYT and insertion of A20. 2) Chloramphenicol was cut out using SwaI from the confirmed recombinant plasmid and 3) re-ligated to obtain the desired recombinant plasmid, which will be used to generate the modified adenovirus.

FIG. 6 shows the diagram of generation of mutation of Y477A, deletion of TAYT and insertion of A20 in adenovirus armed with hIL-21. 1) Mutation of Y477A, deletion of TAYT and insertion of A20 were achieved by combination of shuttle cassette and backbone virus genome in the vector through homologous recombination. The resulted recombinant was selected and grown under Chloramphenicol on LB plate, colonies were picked up, grown and plasmid was extracted, then subject to sequencing to confirm the mutation of Y477A, deletion of TAYT and insertion of A20. 2) Chloramphenicol was cut out using SwaI from the confirmed recombinant plasmid and 3) re-ligated to obtain the desired recombinant plasmid, which will be used to generate the modified adenovirus.

FIG. 7 shows the sequence of the cassette for the deletion of E3gp19k.

FIG. 8 shows the sequence of the cassette for insertion of human IL-21 into the E3gp19k region by replacing E3gp19k.

FIG. 9 shows the sequence of the cassette for the mutation of Y477A, deletion of TAYT and insertion of RDG peptide A20 into the fibre region.

FIG. 10 shows the sequencing result of E3gp19k deletion of control virus construct pAd-c. E3gp19k was deleted between ATGA (28372) and TTTACT (29212) as shown in the alignment of the top panel. The sequence, CCCATCATTTGAAGCTTCAAATTACGGG, was inserted between ATGA (28372) and TTTACT (29212) after the removal of Chloroform using SwaI restriction enzyme then filled in with a linker sequence.

FIG. 11 shows the modification of control virus construct pAd-c. Sequencing result shows the mutation of Y477A, deletion of TAYT.

FIG. 12 shows the modification of control virus construct Ad-c in the fibre region by insertion of RGD sequence A20. The sequence of A20: AACGCAGTACCTAACTTGAGA GGAGATCTACAGGTGTTGGCACAGAAGGTCGCACGTACT

FIG. 13 shows the sequencing result of removal of Chloroform using SwaI restriction enzyme in control virus construct Ad-c.

FIG. 14 shows that human IL-21 was inserted in E3gp 19K region in virus construct pAd-IL21. Human IL-21 was inserted between ATGA and ATAAT of Adenovirus genome.

FIG. 15 shows the sequencing result of removal of Chloroform using SwaI restriction enzyme in virus Ad-IL21.ATAAT is the extra sequence left in the virus genome.

FIG. 16 shows the modification of virus construct pAd-IL21. Sequencing result shows the mutation of Y477A, deletion of TAYT.

FIG. 17 shows the modification of virus construct pAd-IL21 in the fibre region by insertion of RGD sequence A20.

FIG. 18 shows the sequencing result of removal of Chloroform using SwaI restriction enzyme in virus construct pAd-IL21. ATTTAAAT is the extra sequence left in the virus genome.

FIG. 19 shows the expression of human IL-21 by modified adenovirus. Cell culture medium was collected from 293T cells infected with modified adenoviruses, human IL21 in the cell culture medium was measured by ELISA.

FIG. 20 shows that the modified virus Ad5 of the present application is able to specifically target and kill a tumor cell.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “Ad5” herein generally refers to a kind of human adenoviruses (HAdV/Ad), and may be also named as human adenoviruses species C type 5, or HAdV-05. HAdV-05 is a pathogen that may cause respiratory symptoms of variable severity including acute, mild, and none, i.e., asymptomatic (Echavarria, 2009, Edwards et al., 1985, Fox et al., 1969, Garnett et al., 2009). The Ad5 has been commonly used for gene transfer experiments given its ability to infect a wide group of different cell types and the capacity to harbor large genes in their genome incorporated via homologous recombination techniques.

The term “cytokine” herein generally refers to a general class of biological molecules which may affect cells of the immune system. The cytokine may comprise biological molecules that act locally or may circulate in the blood to regulate or modulate an individuals immune response to cancer. For example, the cytokine may comprise interferon-alpha (IFN-α), interferon-beta (IFN-β), and interferon-gamma (IFN-γ), interleukins (e.g., IL-1 to IL-29, in particular, IL-2, IL-5, IL-6, IL-7, IL-10, IL-12, IL-15 and IL-18), tumor necrosis factors (e.g., TNF-alpha and TNF-beta), erythropoietin (EPO), MIP3a, monocyte chemotactic protein (MCP)-1, intracellular adhesion molecule (ICAM), macrophage colony stimulating factor (M-CSF), granulocyte colony stimulating factor (G-CSF) and granulocyte-macrophage colony stimulating factor (GM-CSF).

The term “IL-21” herein generally refers to a pleiotropic cytokine with actions on a broad range of lymphoid, myeloid and epithelial cells. The IL-21 may have a key role in B cell differentiation to plasma cells and in the development of T follicular helper cells, promoting functional germinal centers and immunoglobulin production. For example, the IL-21 may induce a functional programme in CD8⁺ T cells that leads to enhanced survival, antiviral activity and anti-tumor activity. The IL-21 may regulate both innate and adaptive immune responses, and it may have key roles in anti-tumor as well as the development of autoimmune diseases and inflammatory disorders. The Gene ID of human IL-21 may be 59067.

The term “A20” herein generally refers to an A20FMDV2 peptide. The A20 may be derived from foot-and-mouth disease virus. The A20 may have an amino acid sequence as set forth in SEQ ID No. 5: (NAVPNLRGDLQVLAQKVART). The A20 may exhibit high selectivity and affinity for the tumor-related αvβ6 integrin.

The term “fibre region” herein generally refers to the fibre structure of the adenoviruses (Ad). The Ad may have a capsid consisting of three main exposed structural proteins, the hexon, fiber, and penton base. The primary role of the fiber region may be the tethering of the viral capsid to the cell surface via its interaction with a cellular receptor. The fiber region may have: A N-terminal tail, a central shaft made of repeating sequences, and a C-terminal globular knob domain. The first 45 residues of the fiber may be highly conserved among different serotypes. The mutation of the fibre region may be referred to Table 2 of “The influence of adenovirus fiber structure and function on vector development for gene therapy”.

The term “E1ACR2 gene” herein generally refers to a gene of the Ad5. Various mutants with a deletion of E1ACR2 may be highly efficacious in preclinical studies (Cancer Res. 2002 Oct. 15; 62(20):5736-42). The E1ACR2 encoded by the E1ACR2 gene may be responsible for binding and inactivation of pRb thereby releasing E2F for S-phase induction. And E1ACR2 may improve safety in vivo but also promote cell death in response to cytoxic drug-induced apoptosis.

The term “E1B19K gene” herein generally refers to a gene of the Ad5. The E1B19K encoded by the E1B19K gene may promote viral replication and spread by blocking Bax-Bak oligomerization and mitochondrial pore-formation analogous to the cellular Bcl-2 homologue. And ΔE1B19K-mutants may have increased therapeutic index and lower liver toxicity in vivo (Clin Cancer Res. 2010 Jan. 15; 16(2): 541-553).

The term “E3gp19K gene” herein generally refers to a gene of the Ad5. The E3gp19K encoded by the E3gp19K gene is a transmembrane glycoprotein, and may prevent cytolysis by cytotoxic T lymphocytes (CTL). Deletion of the E3gp19K gene may promote tumor antigen presentation and stimulate an immune response that targets both infected and noninfected cancer cells, which may be as advantage for tumor-mediated immune checkpoint inhibition (Oncolytic Virother. 2016; 5: 45-57).

The term “gene editing” herein generally refers to a type of genetic engineering in which DNA is inserted, deleted, modified or replaced in the genome of a living organism. In the present application, the gene editing may be performed using enzymes, for example nucleases that have been engineered to target a specific DNA sequence, where they may introduce cuts into the DNA strands, enabling the removal of existing DNA and the insertion of replacement DNA. The gene editing may be performed by CRISPR/Cas system.

The term “gene recombination” herein generally refers to the exchange of genetic material either between multiple chromosomes and/or between different regions of the same chromosome. The gene recombination may be mediated by homology; that is, homologous regions of chromosomes line up in preparation for exchange, and some degree of sequence identity may be required.

The term “αvβ6 integrin” herein generally refers to an epithelial-specific integrin that is a receptor for the extracellular matrix (ECM) proteins fibronectin, vitronectin, tenascin and the latency associated peptide (LAP) of TGF-β. The αvβ6 integrin may actually promote carcinoma progression. The αvβ6 integrin may be highly upregulated in carcinomas of the breast, lung, oral and skin squamous cell carcinomas (SCC), colon, stomach and endometrium among others.

An embodiment of the present application provides a sequence, comprising at least one of:

a sequence set forth in SEQ ID NO:1;

a sequence set forth in SEQ ID NO:2;

a sequence set forth in SEQ ID NO:3;

a sequence set forth in SEQ ID NO:4;

partially or completely deletion of E1ACR2;

partially or completely deletion of E1B19k;

partially or completely deletion of E3gp19k;

IL-21;

Mutant Ad5 fibre protein;

Ligand of αvβ6 integrin;

therapeutic gene or a modified version thereof; or

Ligands or antibodies that target T cells.

An embodiment of the present application provides a virus, comprising at least one of:

a sequence set forth in SEQ ID NO:1;

a sequence set forth in SEQ ID NO:2;

a sequence set forth in SEQ ID NO:3;

a sequence set forth in SEQ ID NO:4;

partially or completely deletion of E1ACR2;

partially or completely deletion of E1B19k;

partially or completely deletion of E3gp19k;

IL-21;

Mutant Ad5 fibre protein;

Ligand of αvβ6 integrin;

therapeutic gene or a modified version thereof; or

Ligands or antibodies that target T cells.

An embodiment of the present application provides a sequence, comprising at least one of:

a portion or all of the E1ACR2, E1B19k, or E3gp19k is replaced by IL-21;

a portion or all of the E1ACR2, E1B19k, or E3gp19k is replaced by mutant Ad5 fibre protein;

a portion or all of the E1ACR2, E1B19k, or E3gp19k is replaced by ligand of αvβ6 integrin;

a portion or all of the E1ACR2, E1B19k, or E3gp19k is replaced by a sequence set forth in SEQ ID NO: 4;

a portion or all of the E1ACR2, E1B19k, or E3gp19k is replaced by a ligand or an antibody that targets T cells;

a portion or all of the E1ACR2, E1B19k, or E3gp19k is replaced by a tumor targeting gene;

or

a portion or all of the E1ACR2, E1B19k, or E3gp19k is replaced by a therapeutic gene or a modified version thereof.

An embodiment of the present application provides a virus, comprising at least one of:

a portion or all of the E1ACR2, E1B19k, or E3gp19k is replaced by IL-21;

a portion or all of the E1ACR2, E1B19k, or E3gp19k is replaced by mutant Ad5 fibre protein;

a portion or all of the E1ACR2, E1B19k, or E3gp19k is replaced by ligand of αvβ6 integrin;

a portion or all of the E1ACR2, E1B19k, or E3gp19k is replaced by a sequence set forth in SEQ ID NO: 4;

a portion or all of the E1ACR2, E1B19k, or E3gp19k is replaced by a ligand or an antibody that targets T cells;

a portion or all of the E1ACR2, E1B19k, or E3gp19k is replaced by a tumor targeting gene;

or

a portion or all of the E1ACR2, E1B19k, or E3gp19k is replaced by a therapeutic gene or a modified version thereof.

In the above embodiments of the present application, the Ad5 fibre protein can include a mutation at Y477A and a deletion of TAYT. The ligand of αvβ6 integrin can be a peptide that selectively binds through an Arg-Gly-Asp (RGD)-domain to αvβ6.

In the above embodiments of the present application, the virus can be adenovirus, especially adenovirus type 5.

In the above embodiments of the present application, the sequence may further comprise therapeutic genes including immunomodulators, immune co-stimulatory pathway activating molecules, checkpoint inhibitors, cytotoxic genes, tumor suppressor genes, anti-angiogenesis genes, etc.

The immunomodulator genes may include cytokine genes, such as: IL12, IL21, IL2, IL15, IL8 or a modified version of any of these.

The immune co-stimulatory pathway activating molecules may include gene encodes CD40 ligand (CD40L), ICOS ligand, GITR ligand, 4-1BB ligand, OX40 ligand, TL1A, CD30 ligand, CD27 or Flt3 ligand or a modified version of any of these

The checkpoint inhibitors may include PD-1 inhibitor, PD-L1 inhibitor, CTLA-4 inhibitor or a modified version of any of these.

The tumor suppressor genes may include HIC1, etc. or a modified version of any of these.

The genes used in the present application can be obtained from NCBI genebank.

An embodiment of the present application provides an expression vector or a host cell comprising any sequence described hereabove.

Treatment Strategies

An embodiment of the present application provides a virus used for a method of treating the human or animal body, comprising at least one of:

used alone as monotherapy; or

used in combination with one or more medicament.

The medicament of the embodiments of the present application can be known anticancer agents, inhibitors, agonists, antagonists, chemotherapeutic agents, radiation agents, especially PI3K6 inhibitors or immune checkpoint inhibitors

An embodiment of the present application provides a virus used for use in the manufacture of a medicament for treating the human or animal body.

An embodiment of the present application provides a virus used for use in inducing cancer cells death, regulating a biological activity of the cancer cells, regulating immune response, enhancing proliferation and/or cytotoxicity of T cells.

An embodiment of the present application provides a virus used for use in the manufacture of a medicament for suppressing cancer cells growth, inducing cancer cells death, and/or regulating a biological activity of the cancer cells.

The biological activity of the cancer cells comprises inhibition of cancer cells replication, inhibition of cancer cells division, inhibition of DNA repair of cancer cells, inhibition of cancer cells migration, or promote cancer death.

An embodiment of the present application provides a product of manufacture comprising a virus in a sterile vial, ampoule or syringe.

An embodiment of the present application provides a pharmaceutical composition comprising a virus of the embodiments of the present application.

In an embodiment of the present application, the pharmaceutical composition further comprises an anti-cancer agent and/or antibody.

In an embodiment of the present application, the pharmaceutical composition further comprises a pharmaceutically acceptable carrier, a diluent, and/or an excipient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

Suitable routes of administration may, for example, include intratumoral, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

An embodiment of the present application provides a therapeutic method for a disease, comprising administering an effective amount of a sequence, expression vector, host cell, virus, pharmaceutical composition, or medicament.

Exemplary diseases include cancers, proliferative diseases, autoimmune diseases, etc.

In one aspect, the present application provides a modified virus Ad5, wherein the modified virus Ad5 is capable of expressing a cytokine, and the modified virus Ad5 is capable of expressing an A20.

In the present application, it is found that compared to the virus Ad5 which is not capable of expressing the cytokine of the present application, the modified virus Ad5 in the present application may have an enhanced ability in adjusting immune reaction activity of an immune cell (for example, a T cell, a NK cell).

In the present application, it is found that compared to the virus Ad5 which is not capable of expressing the A20 of the present application, the modified virus Ad5 in the present application may have an enhanced ability in targeting a tumor cell and/or killing a tumor cell. For example, compared to the virus Ad5 which is capable of expressing a protein targeting the integrin other than the A20 of the present application, the modified virus Ad5 in the present application may have an enhanced ability in targeting a tumor cell and/or killing a tumor cell. For example, compared to the virus Ad5 which is capable of expressing a protein targeting other targets in the tumor micro environment, the modified virus Ad5 in the present application may have an enhanced ability in targeting a tumor cell and/or killing a tumor cell. In the present application, the tumor may comprise the cancer in the present application.

For example, the cytokine may be originated from human.

For example, the cytokine may comprise an interleukin, a tumor necrosis factor, an interferon, a chemokine, a lymphokine and/or a growth factor.

For example, the cytokine may comprise an IL12, an IL2, an IL15 and/or an IL8.

For example, the cytokine may comprise an IL-21.

In the present application, it is found that compared to the virus Ad5 which is capable of expressing a cytokine other than the IL-21, the modified virus Ad5 of the present application expressing the IL-21 may have significantly less toxicity to the subject administrated the virus Ad5. For example, the toxicity may be measured in vivo in an animal model. For example, the body weight of the administrated animal in the animal model may be used to illustrate the degree of the toxicity.

For example, a gene encoding the cytokine may be incorporated in to the genome of the modified virus Ad5.

For example, the A20 may be originated from a foot-and mouth disease virus (FMDV).

For example, the gene encoding the A20 may have a nucleic acid sequence as set forth in SEQ ID NO. 4. For example, the A20 may have an amino acid sequence as set forth in SEQ ID NO. 5.

For example, a gene encoding the A20 may be incorporated in to the genome of the modified virus Ad5. In the present application, the gene encoding the A20 may be incorporated in to anywhere of the genome of the modified virus Ad5, as long as the endogenous promoter of the modified virus Ad5 may be used to express the A20. For example, the gene encoding the A20 may be incorporated in to the site where the original gene (for example, the E1ACR2 gene, the E1B19K gene or the E3gp19K gene) is to be deleted.

For example, a gene encoding the A20 may be incorporated in to the HI-loop of the modified virus Ad5.

For example, the incorporation may use a method of gene editing and/or gene recombination.

For example, the modified virus Ad5 may have at least one modification in fibre region.

For example, the modification in fibre region may comprise an amino acid substitution Y477A.

For example, the modification in fibre region may comprise a deletion of amino acids TATY at the residues 489-492.

In the present application, the modification in fibre region may be consisted of an amino acid substitution Y477A and a deletion of amino acids TATY at the residues 489-492. For example, the TATY at the residues 489-492 may mean the 489th-492th amino acid residues from the N terminal of the fibre region.

For example, compared to a wild virus Ad5, the expression and/or activity of a E1ACR2 gene may be downregulated in the modified virus Ad5.

For example, compared to a wild virus Ad5, the expression and/or activity of a E1B19K gene may be downregulated in the modified virus Ad5.

For example, compared to a wild virus Ad5, the expression and/or activity of a E3gp19K gene may be downregulated in the modified virus Ad5.

For example, compared to a wild virus Ad5, the expression and/or activity of the E1ACR2 gene, the E1B19K gene and the E3gp19K gene may be downregulated in the modified virus Ad5. For example, the expression level of the E1ACR2 gene, the E1B19K gene and the E3gp19K gene may be downregulated significantly or nearly hardly to be detected in the modified virus Ad5. For example, the expression level of E1ACR2, E1B19K and E3gp19K may be downregulated significantly or nearly hardly to be detected in the modified virus Ad5. For example, the activity and/or the function of E1ACR2, E1B19K and E3gp19K may be downregulated significantly or nearly hardly to be detected in the modified virus Ad5.

For example, the downregulation may use a method of gene editing and/or gene recombination.

For example, the gene editing may use an anti-sense RNA, a siRNA, a shRNA and/or a CRISPR/Cas system. For example, the gene editing may use a CRISPR/Cas9 system.

For example, at least a portion of the genes encoding the E1ACR2 gene, at least a portion of the E1B19K gene and at least a portion of the E3gp19K may be deleted.

For example, the gene encoding a cytokine may be incorporated in to the site of the E1ACR2 gene, the E1B19K gene or the E3gp19K gene.

For example, in the modified virus Ad5, the E1ACR2 gene, the E1B19K gene and the E3gp19K gene may be deleted, and the gene encoding a cytokine and the gene encoding the A20 may be incorporated.

For example, in the modified virus Ad5, the E1ACR2 gene, the E1B19K gene and the E3gp19K gene may be deleted, and the gene encoding an IL-21 (for example, a human IL-21) and the gene encoding the A20 may be incorporated.

For example, in the modified virus Ad5, the E1ACR2 gene, the E1B19K gene and the E3gp19K gene may be deleted, and the gene encoding an IL-21 (for example, a human IL-21) and the gene encoding the A20 may be incorporated, and the modification in fibre region thereof may be consisted of an amino acid substitution Y477A and a deletion of amino acids TAYT at the residues 489-492. For example, the modified virus Ad5 may be named as KMAd1.

For example, the gene encoding an IL-21 may be incorporated in the original site of the E3gp19K gene.

In the present application, the modified virus Ad5 may be capable of expressing an exogenous gene and/or an exogenous protein. For example, the modified virus Ad5 may be capable of expressing a gene and/or a ligand targeting a T cell, a gene and/or a ligand targeting a tumor cell, and/or a therapeutic gene.

For example, the therapeutic gene may be selected from a group consisting of: a gene encoding an immune co-stimulatory pathway activating molecule, a gene encoding a checkpoint inhibitor, a gene encoding a cytotoxic, a gene encoding a tumor suppressor gene, and an anti-angiogenesis gene.

For example, the immune co-stimulatory pathway activating molecule may be selected from a group consisting of: a CD40 ligand (CD40L), a ICOS ligand, a GITR ligand, a 4-1BB ligand, an OX40 ligand, a TL1A, a CD30 ligand, a CD27, and a Flt3 ligand or the variant thereof.

For example, the checkpoint inhibitor may be selected from a group consisting of: a PD-1 inhibitor, a PD-L1 inhibitor and a CTLA-4 inhibitor.

For example, the tumor suppressor gene may comprise HIC1 gene.

In another aspect, the present application provides an isolated nucleic acid molecule, encoding the modified virus Ad5 of the present application.

The isolated nucleic acid or isolated nucleic acids may be synthesized using recombinant techniques well known in the art. For example, the isolated nucleic acid or isolated nucleic acids may be synthesized with an automated DNA synthesizer. Standard recombinant DNA and molecular cloning techniques include those described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T J. Silhavy, M L. Bennan, and L. W. Enquist, Experiments with Gene Fusions, Cold Spring Harbor Laboratory, Cold Spring Harbor, N.Y. (1984) and by Ausubel, F. M et al., Current Protocols in Molecular Biology, pub. by Greene Publishing Assoc. and Wiley-Interscience (1987). Briefly, the subject nucleic acids may be prepared from genomic DNA fragments, cDNAs, and RNAs, all of which may be extracted directly from a cell or recombinantly produced by various amplification processes including but not limited to PCR and RT-PCR.

In another aspect, the present application provides a vector, comprising the modified virus Ad5 of the present application, and/or the isolated nucleic acid molecule of the present application.

An expression vector may be suitable for use in particular types of host cells and not others. For example, the expression vector can be introduced into the host organism, which is then monitored for viability and expression of any genes/polynucleotides contained in the vector. The expression vector may also contain one or more selectable marker genes that, upon expression, confer one or more phenotypic traits useful for selecting or otherwise identifying host cells that carry the expression vector.

In another aspect, the present application provides a cell, comprising the modified virus Ad5 of the present application, the isolated nucleic acid molecule of the present application, and/or the vector of the present application.

The cell may be a eukaryotic cell or a prokaryotic cell.

In another aspect, the present application provides a pharmaceutical composition, wherein the pharmaceutical composition may comprise the modified virus Ad5 of the present application and a pharmaceutically accepted adjuvant.

In another aspect, the present application provides a kit comprising the modified virus Ad5 of the present application.

The pharmaceutical composition may, for example, be in a form suitable for administration. The pharmaceutical compositions of the present application may comprise a therapeutically effective amount of the modified virus Ad5 of the present application.

In the present application, the pharmaceutical accepted adjuvant may comprise detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and/or mixtures thereof.

In another aspect, the present application provides a method of treating a disease and/or a disorder, comprising administrating the modified virus Ad5 of the present application, the isolated nucleic acid molecule of the present application, the vector of the present application, the cell of the present application, and/or the pharmaceutical composition of the present application to a subject in need of.

In another aspect, the present application provides the modified virus Ad5 of the present application, the isolated nucleic acid molecule of the present application, the vector of the present application, the cell of the present application, and/or the pharmaceutical composition of the present application, in a use of treating a disease and/or a disorder.

In another aspect, the present application provides the modified virus Ad5 of the present application, the isolated nucleic acid molecule of the present application, the vector of the present application, the cell of the present application, and/or the pharmaceutical composition of the present application in preparing a medicament, and the medicament is for treating a disease and/or a disorder.

For example, the method may comprise administrating the modified virus Ad5 of the present application to a subject in need of in a combination of at least one agent, and the agent is selected from a group consisting of an anti-cancer agent, an agonist, an antagonist, a chemotherapeutic agent and a radiation agent.

For example, the disease may comprise a tumor.

For example, the disease may comprise a tumor expressing αvβ6 integrin.

For example, the disease may comprise a pancreatic cancer, a head and neck cancer, and/or an ovarian cancer.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s. c., sub cutaneous(ly); and the like.

Product Design and Construction

1. Use pAd2D as the backbone to generate further modified Ad5 virus.

pAd2D plasmid has two modifications, namely E1ACR2 deletion and E1B19k deletion.

2. Deletion of E3Bgp19k in pAd2D plasmid

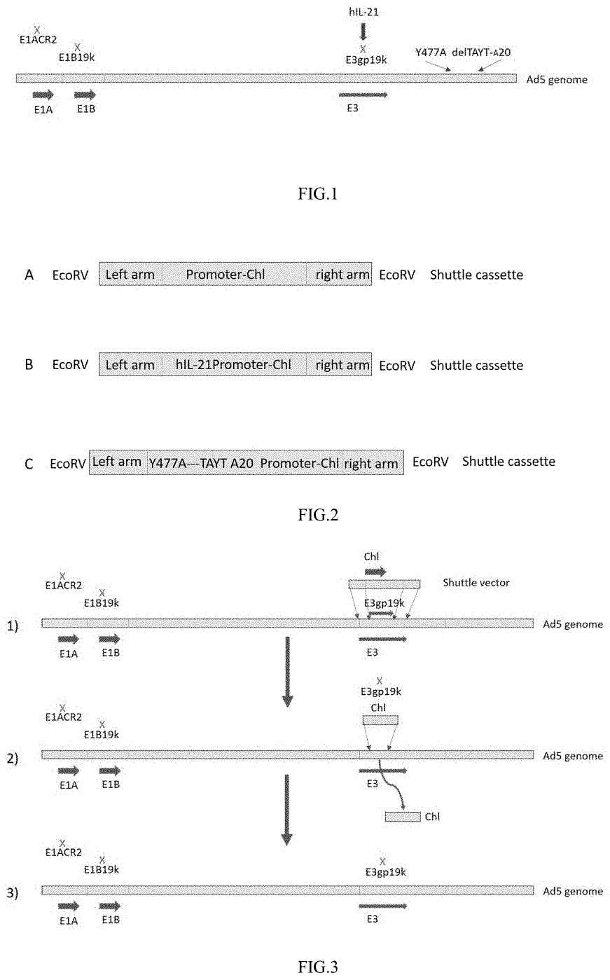

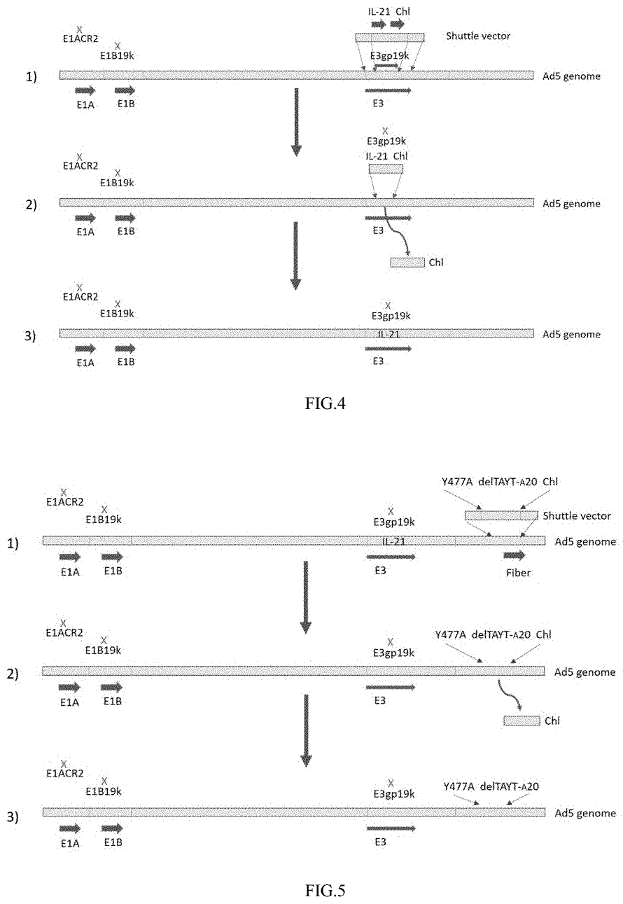

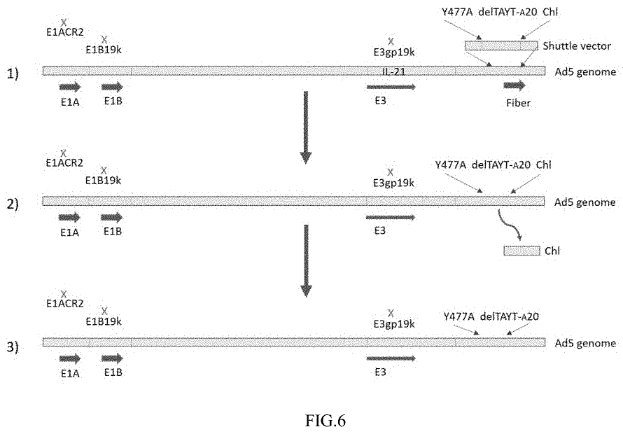

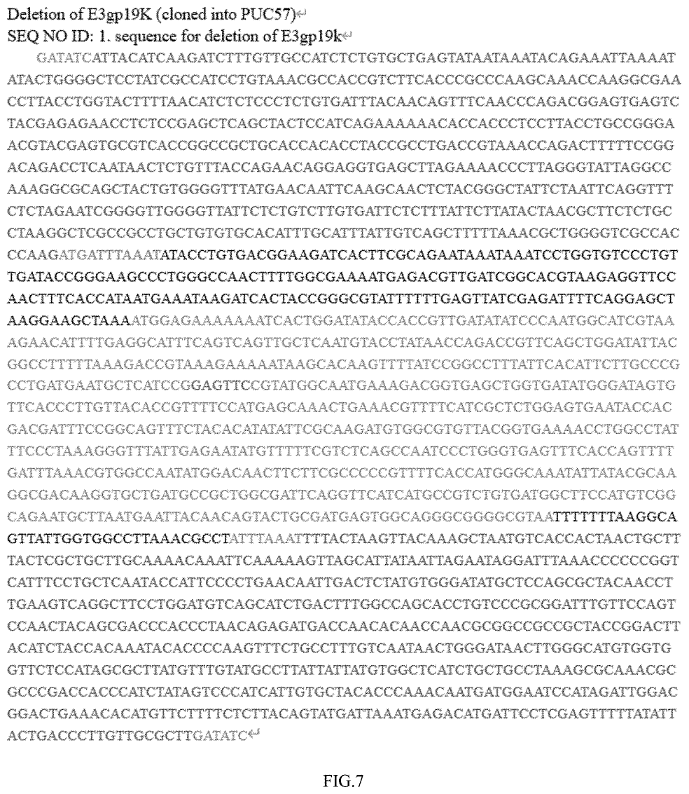

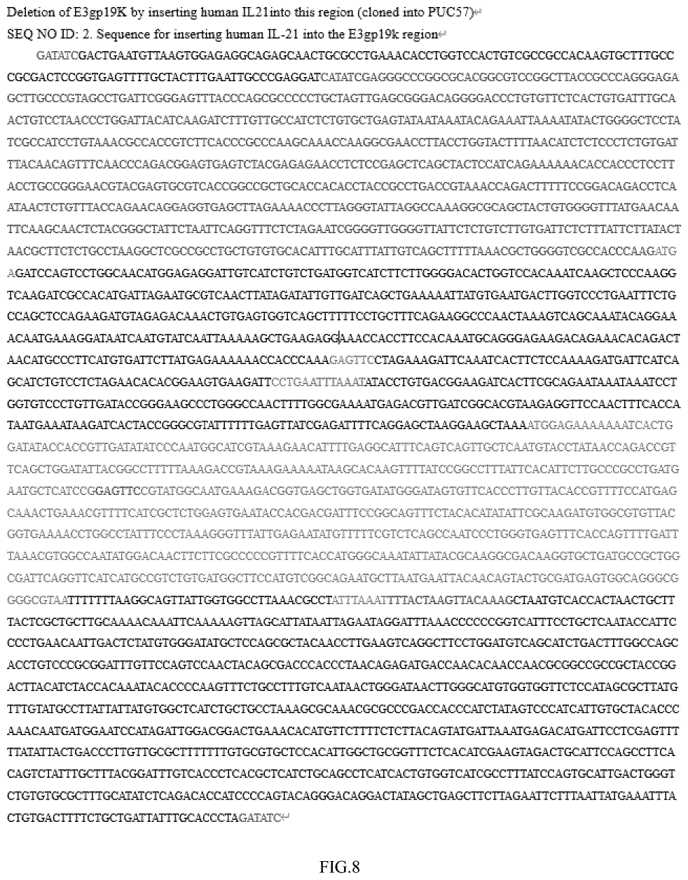

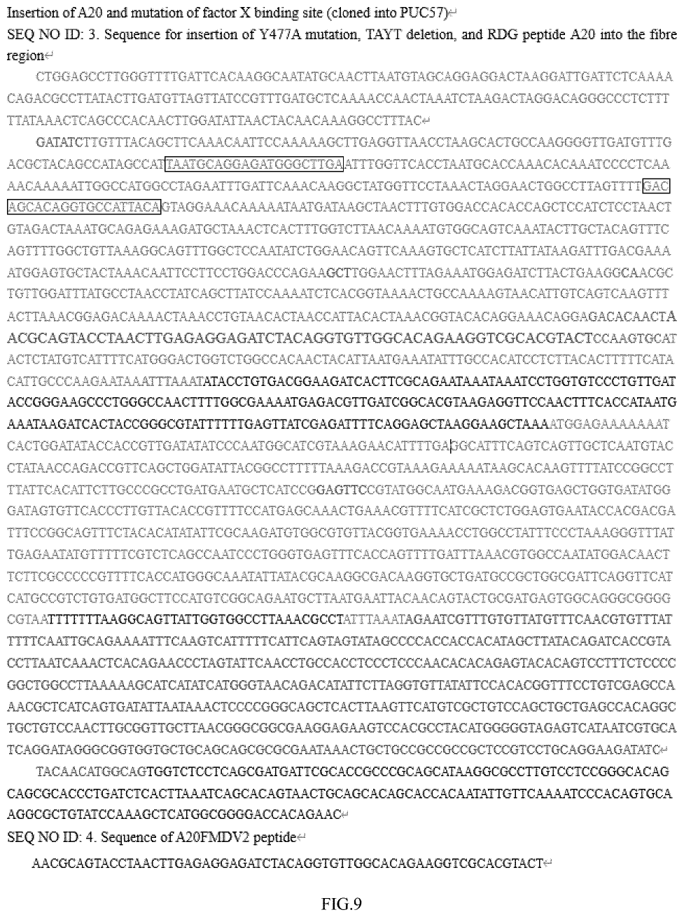

The cassette is designed as follows (see FIG. 3, 4 for a schematic diagram):

Left arm-promoter-Chloramphenicol-right arm

2. Human interleukin 21 (hIL-21) and Chloramphenicol integration into the E3Bgp19k region

The cassette is designed as follows (see FIG. 3, 4 for a schematic diagram):

Left arm—hIL-21-promoter-Chloramphenicol—right arm

3. Mutation of Y477A and deletion of TAYT in fiber region

The cassette is designed as follows (see FIG. 5 for a schematic diagram):

Fiber region with Y477A mutation and TAYT deletion-promoter-Chloramphenicol

4. The structure of the obtained final product is shown in FIG. 1:

Materials and Method:

Cell lines: All tumor cell lines used were from the ATCC or provided by a collaborator. All human cancer cell lines were genotyped by STR assay. The murine tumor cell lines used in this study included: the colorectal cancer cell line MC38 was derived from C57B/6 mice.

Backbone Virus gene: Plasmid pAd2D with deletions of E1ACR2 and E1B19k was gift from a collaborator.

Construction of the pS-E3gp19K shuttle vector:

The pS-E3gp19K shuttle vector includes the E3gp19K left arm targeting the left side of the E3gp19K gene and the E3gp19K right arm targeting the right side of the E3gp19K gene. The chloramphenicol gene with its promoter is located between the E3gp19K left arm and right arm. All of the above sequences were spliced together and synthesized by the company and cloned into the ECoRV sites of PUC57 vector (See in FIG. 2A).

Construction of the pS-E31L21 shuttle vector:

The pS-E31L21 shuttle vector includes the E3gp19K left arm targeting the left side of the E3gp19K gene and the E3gp19K right arm targeting the right side of the E3gp19K gene. The human IL-21 gene and the chloramphenicol gene with its promoter is located between the E3gp19K left arm and right arm. The human IL-21 gene is under the promotor of E3gp19K. All of the above sequences were spliced together and synthesized by the company and cloned into the ECoRV sites of PUC57 vector (See in FIG. 2B).

Construction of the pS-A20 shuttle vector:

The pS-A20 shuttle vector includes the fibre gene with mutation at Y477A, deletion of TAYT and A20 insertion. All of the above sequences were spliced together and synthesized by the company and cloned into the ECoRV sites of PUC57 vector (See in FIG. 2C).

Homologous Recombination:

Electrocompetent E. coli BJ5183 cells were used for homologous recombination. Recombination shuttle cassette fragment was realized by ECoRV restriction enzyme from respective PUC57 based construct and purified from agarose gel. pAd2D and the linearized shuttle cassette fragment were transferred into twenty microliters of electrocompetent BJ5183 cells by electroporation, and electroporation was performed in 2.0 mm cuvettes at 2,500 V, 200 ohms, and 25 μL in a Bio-Rad Gene Pulser electroporator. The cells were immediately placed in 500 μl of LB-Broth and grown at 37° C. for 20 min. One hundred twenty-five microliters of the cell suspension were then inoculated onto each of four 10-cm Petri dishes containing L-agar plus 25 μg/ml of chloramphenicol. After 16-20 hr growth at 37° C., 10-25 colonies per dish generally were obtained. The smaller colonies (which usually represented the recombinants) were picked and grown in 2 ml of 1-Broth containing 25 μg/ml of chloramphenicol. Plasmid was extracted using the miniprep kit.

Expansion of Recombinant Plasmid

bug of plasmid extracted from BJ5183 cells was transformed into Top10 chemically competent cells containing 25 μg/ml of chloramphenicol, and grow for 18 hrs before extraction of plasmid from the bacterial.

Obtain Recombinant Plasmid without Chloramphenicol

Chloramphenicol was released from the recombinant plasmid using SwaI restriction enzyme, then purified the large fragment of recombinant, re-ligated before transformed into top 10 competent cells, the cells were grown in LB-broth for 18 h before plasmid extraction.

Confirmation of Gene Modification

The gene modification in the recombinant plasmid was confirmed by DNA sequencing using respective primer. E3 sequencing primer:5′-GGGTTGGGGTTATTCTCT-3′(SEQ ID No. 6), fibre region sequencing primer:5′-GACAGCACAGGTGCCATTACA3′(SEQ ID No. 7).

Packaging Adenovirus

Adenovirus genome was realized from the plasmid using PacI restriction enzyme, and purified from agarose gel. 2 μg of linearized adenovirus genome was transformed into one well of 293T cells in 6-well plate using Effectene transfection reagent according to the manufacturers instructions. The transformed 293T cells was placed into cell culture incubator for 10 days for the adenovirus appearing.

Amplification of the Virus:

Once the recombinant virus was confirmed to be the desired recombinant virus, 50 μl of the virus lysate was added to a T175 flask containing 293Tcells, and grown to 80-90% confluence in a cell culture medium containing about 30 ml. After 48 hours, the cells and medium were scraped off and “primary virus amplification” was saved.

Large-Scale Virus Production:

The primary virus amplification from above was rapidly frozen and thawed once and diluted to the volume required for cell culture required to infect 36 T175 flasks (80-90% confluence) containing 293T cells. After 48 hours, infected 293T cells were harvested by scraping and collected by repeating centrifugation at 2,000 rpm (4° C.) for several rounds. The precipitate was washed in PBS, resuspended in 12 ml of 10 mM Tris-HCl (pH 9) buffer and stored at −80° C. for later purification.

Purification of Adenovirus

As described before. Defrost the viral concentrate at 37° C., then freeze/thaw it a further 2 times by transferring the sample between liquid nitrogen and a 37° C. water bath. Spin the virus suspension for 10 min at 6000 rpm/room temp. Transfer the supernatant from the centrifuge tube into a 50 ml tube and lay supernatant onto the CsCl for banding immediately. Once balanced, spin at 25,000 rpm for 2 hours at 15° C. The virus should band between the CsCl steps. Normally three bands are visible; the highest is cellular debris, the middle band empty Adenoviral particles, and the lowest band successfully encapsulated infectious particles. The ultracentrifuge tube is placed in a clamp (preferably with blue clamps so it is easier to see the virus band), above a beaker containing Vikron. The tube is then pierced using a 19G needle fitted to a 10 ml syringe, just below the lowest band (approx. 1 cm below), taking care to only pierce one of its sides. The viral band is then carefully removed in the minimum amount of CsCl, and transferred to a labelled 15 ml tube. Once all the bands have been pooled, they are then layered onto 2.5 ml of a 1.35 g/ml CsCl solution in a 1/2×2″ centrifuge tube (small Beckman tubes). Depending on the total pooled volume, this can be split equally between two or three ultracentrifuge tubes. These tubes are balanced as before, prior to spinning for 40,000 rpm at 15° C. for 15 hours (overnight) using a combination of a Beckman SW55ti swing out rotor in an Optima LE-80K ultracentrifuge. The virus band (which should be located at the tube center) is collected as before, then transferred to a labelled 15 ml tube. The volume is then made up to 12 ml with TSG (roughly a 2-3-fold dilution), if small volume of virus only top up to 9 ml. Use a new needle and a new syringe for every tube, which has been spun. The virus/TSG mixture is then injected into a slide-a-lyzer (pink dialysis cassette) using the 18G (green tipped) needle supplied and the 20 ml syringes. Transfer virus from 12 ml tube into small bijou beaker, syringe is otherwise too big. It is also necessary to remove excess air from the slide-a-lyzer as the virus is injected, which is done using a syringe placed in one of the three remaining injection ports. Each port may only be used once; therefore, it is necessary to mark each port as used. Once all the harvested virus has been injected carefully remove the syringes and discard into an autoclavable sharps bins. The clear membrane surrounding the virus is semi-permeable so the dialysis buffer can move in and out of the membrane, whereas the virus cant. This step is to place the virus in the right storage buffer. The slide-a-lyzer is then placed in the appropriate size float and transferred to a 51 beaker containing 21 of dialysis solution (see below). The beaker is then placed on a magnetic stirrer in a cold room and the virus left to dialyze for 24 hours. Place slide-a-lyzer in the buffer upside down, with float on top, check if buffer is stirred every now and then. After dialysis the slide-a-lyzer is transferred to a tissue culture hood, the virus is removed using a syringe and transferred to a labelled 15 ml tube (orange cap). The entire virus is aliquoted in lml aliquots, tubes are labelled with the virus name, date (used as batch number), volume and initials. Aliquots are stored in the −80° C. freezer. A small aliquot is later used for virus validation (characterization), to determine the particle count (for TCID50).

Titration of Adenovirus

1×10⁴ 293T cells per well were seeded into 96 well plate. Purified virus was serial diluted with factor 10 till 10-12 dilution. Starting titration, the diluted virus with 10-6 dilution, and add 20 ul into each well of 96-well plate for the whole row of 12 wells. 10-12 dilution was the lowest dilution used for titration.

Enzyme-Linked Immunosorbent Assay:

Expression of hIL-21 was detected by enzyme-linked immunosorbent assay ELISA according to the reagent manufacturers instructions.

Determination of Viral Replication:

Depending on the growth rate, cells were seeded at 2 to 4×10⁵ cells per well in 3 wells of a 6-well plate containing cell culture medium, and infected with 1 PFU/cell of virus the next day. Infected cells and their culture solutions were collected at 24 hours, 48 hours, and 72 hours after infection, respectively. The virus concentration is then determined.

Evaluation of Viral Cytotoxicity In Vitro:

Cells were seeded at 1×10³ and 1×10⁴ cells/well in 96-well plates according to growth rate and infected with virus after 16-18 hours. Cell viability at day 6 after viral infection was determined by MTS assay and EC50 values were calculated as previously described (viral dose killed 50% of tumor cells), all assays were performed at least three times.

In Vivo Efficacy Experiments for Comparing Different Advs:

By subcutaneously injecting 1-5×10⁶ cancer cells, a subcutaneous tumor of the back was established in 10 mice per treatment group and the diameter was 0.4-0.5 cm, and then the mice were regrouped by tumor size and received 1×10⁸ PFU (immune-competent mice) or PBS on days 1, 2, 3, 4, and 5 days. Tumor volume (volume=(length×width 2×π)/6) was measured twice a week until the mice were sacrificed when the tumor area reached 1.69 cm2. The mice used were 4-5 week male mouse strains BALB/c and C57BL/6.

Statistical Analysis:

Comparative statistical analysis was performed using Graphpad Prism 5 unless otherwise stated. Dual condition comparisons were performed using unpaired t-tests. For additional variables of more than one condition, 1 or 2 ANOVA is performed separately. Survival data is represented as a Kaplan-Meier plot with log-rank analysis to plot whether any differences between the groups have statistically significant differences.

Construction Ad5 Mutant with Deletions of Three Regions

The vector pAd2D carrying genome of Ad5 with deletions of E1ACR2 and E1B19k was used as the backbone to delete E3B gp19k gene. The cassette consisting of left arm targeting left side of E1B19k gene, chloramphenicol and right side of E3B gp19k gene was released from cloning vector of PUC57 using restriction enzyme EcoRV and purified from agarose gel. pAd2D and the linearized shuttle fragment were transferred into twenty microliters of electrocompetent E. coli BJ5183 cells by electroporation, and electroporation was performed in 2.0 mm cuvettes at 2,500 V, 200 ohms, and 25 g in a Bio-Rad Gene Pulser electroporator. The cells were immediately placed in 500 μl of LB-Broth and grown at 37° C. for 20 min. One hundred twenty-five microliters of the cell suspension were then inoculated onto each of four 10-cm Petri dishes containing L-agar plus 25 μg/ml of chloramphenicol. After 16-20 hr growth at 37° C., 10-25 colonies per dish generally were obtained. The smaller colonies (which usually represented the recombinants) were picked and grown in 2 ml of 1-Broth containing 25 μg/ml of chloramphenicol. Plasmid was extracted using miniprep kit, and bug plasmid was transformed into Top10 chemically competent cells containing 25 μg/ml of chloramphenicol, and grow for 18 hrs before extraction of plasmid from the bacterial. Chloramphenicol was released from the construct using SwaI restriction enzyme, then purified the large fragment of recombinant, religated before transformed into top 10 competent cells, the cells were grown in LB-broth for 18 h before plasmid extraction. The deletion of E3B gp19k gene in the recombinant was confirmed by DNA sequencing.

Construction Ad5 Mutant with Deletions of Three Regions and Armed with Human IL-21

The vector pAd2D carrying genome of Ad5 with deletions of E1ACR2 and E1B19k was used as the backbone to replace E3B gp19k gene with human IL-21. The cassette consisting of left arm targeting left side of E1B19k gene, human IL-21, chloramphenicol and right side of E3B gp19k gene was released from cloning vector of PUC57 using restriction enzyme EcoRV and purified from agarose gel. pAd2D and the linearized shuttle fragment were transferred into twenty microliters of electrocompetent E. coli BJ5183 cells by electroporation, and electroporation was performed in 2.0 mm cuvettes at 2,500 V, 200 ohms, and 25 μf in a Bio-Rad Gene Pulser electroporator. The cells were immediately placed in 500 μl of LB-Broth and grown at 37° C. for 20 min. One hundred twenty-five microliters of the cell suspension were then inoculated onto each of four 10-cm Petri dishes containing L-agar plus 25 μg/ml of chloramphenicol. After 16-20 hr growth at 37° C., 10-25 colonies per dish generally were obtained. The smaller colonies (which usually represented the recombinants) were picked and grown in 2 ml of 1-Broth containing 25 μg/ml of chloramphenicol. Plasmid was extracted using miniprep kit, and bug of plasmid was transformed into Top10 chemically competent cells containing 25 μg/ml of chloramphenicol, and grow for 18 hrs before extraction of plasmid from the bacterial. Chloramphenicol was released from the construct using SwaI restriction enzyme, then purified the large fragment of recombinant, religated before transformed into top 10 competent cells, the cells were grown in LB-broth for 18 hrs before plasmid extraction. The human IL-21 replacement of E3B gp19k gene in the recombinant was confirmed by DNA sequencing.

Construction Ad5 mutant with deletions of three regions and armed with human IL-21, Y477A, del TAYT, Ad5-3del-A20T, can be also named as KMAd1.

KMAd1 has been conserved in CCTCC on Mar. 25, 2020, with a CCTCC NO. V202024. KMAd1 has been kept in a host cell expressing αvβ6 integrin, e.g. a human pancreatic cancer cell Suit-2. And the human pancreatic cancer cells Suit-2 were cultured in a DMEM cell culture medium comprising 10% fetal bovine serum.

The vector pAd2D carrying genome of Ad5 with deletions of E1ACR2 and E1B19k E3B gp19k replaced by human IL-21 was used as the backbone to construct the recombinant with Y477A, delTAYTA20. The cassette consisting of left arm targeting left side of fibre gene, Y477A mutation, TAYT deletion and A20 peptide, chloramphenicol and right side of E3B gp19k gene was released from cloning vector of PUC57 using restriction enzyme EcoRV and purified from agarose gel. pAd2D and the linearized shuttle fragment were transferred into twenty microliters of electrocompetent E. coli BJ5183 cells by electroporation, and electroporation was performed in 2.0 mm cuvettes at 2,500 V, 200 ohms, and 25 μF in a Bio-Rad Gene Pulser electroporator. The cells were immediately placed in 500 μl of LB-Broth and grown at 37° C. for 20 min. One hundred twenty-five microliters of the cell suspension were then inoculated onto each of four 10-cm Petri dishes containing L-agar plus 25 μg/ml of chloramphenicol. After 16-20 hr growth at 37° C., 10-25 colonies per dish generally were obtained. The smaller colonies (which usually represented the recombinants) were picked and grown in 2 ml of 1-Broth containing 25 μg/ml of chloramphenicol. Plasmid was extracted using miniprep kit, and bug of plasmid was transformed into Top10 chemically competent cells containing 25 μg/ml of chloramphenicol, and grow for 18 hrs before extraction of plasmid from the bacterial. Chloramphenicol was released from the construct using SwaI restriction enzyme, then purified the large fragment of recombinant, religated before transformed into top 10 competent cells, the cells were grown in LB-broth for 18 h before plasmid extraction. The human IL-21 replacement of E3B gp19k gene in the recombinant was confirmed by DNA sequencing.

### EXAMPLES

### Example 1. Confirmation of Deletion of E3gp19k by DNA Sequencing in Control Virus Construct pAd-c

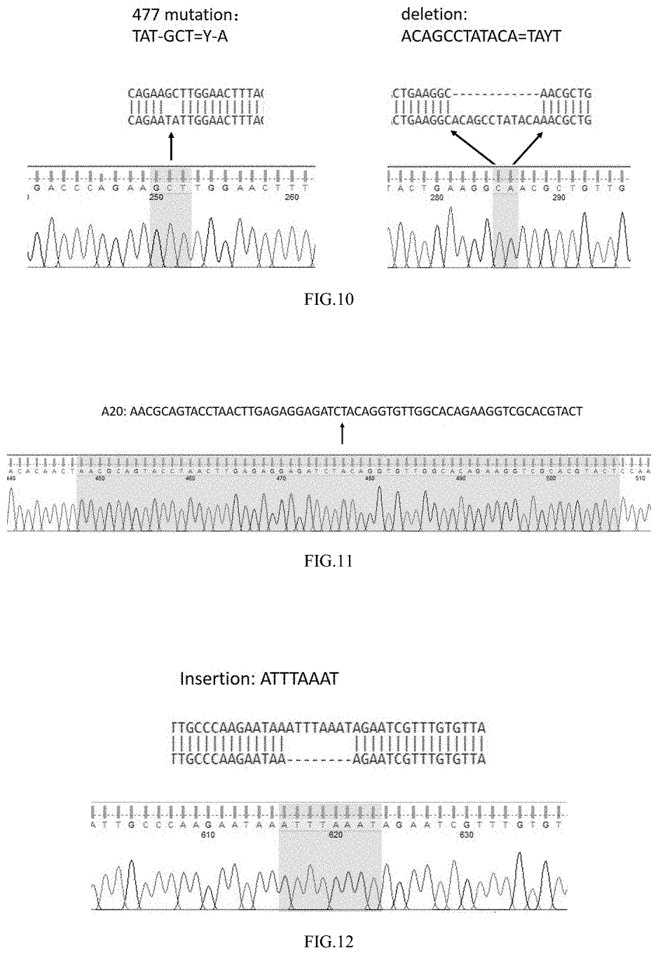

FIG. 10 shows the deletion of E3gp19k in control virus construct pAd-c, which was confirmed by DNA sequencing.

### Example 2. Modification of Control Virus Construct pAd-c

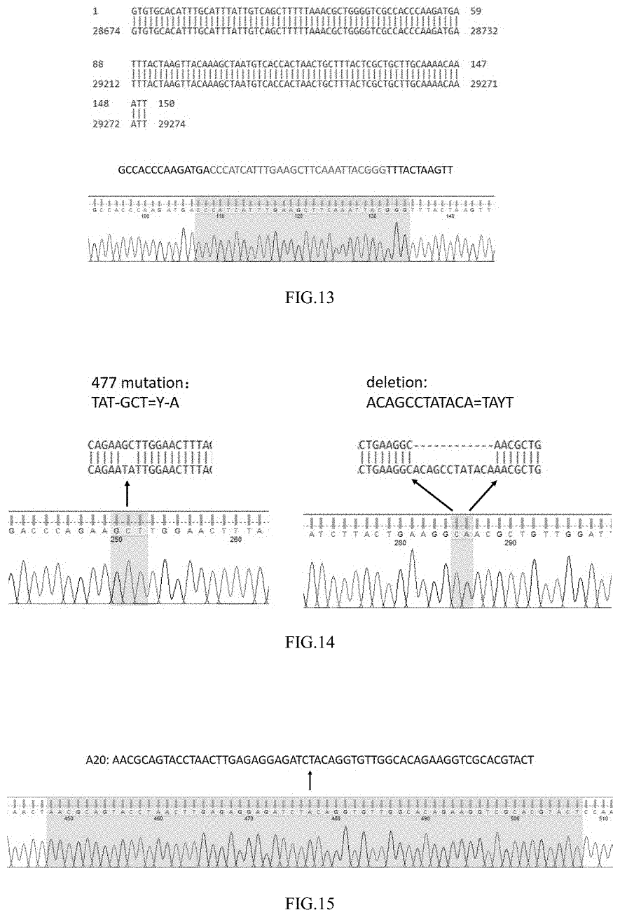

FIG. 11 Sequencing result shows the mutation of Y477A, deletion of TAYT in control virus construct pAd-c. FIG. 13 Sequencing result of removal of Chloroform using SwaI restriction enzyme in control virus construct Ad-c.

### Example 3. Human IL-21 Gene was Inserted into E3gp19k Region of the Adenovirus Genome in Virus Construct pAd3d-hIL21

FIG. 14 shows human IL-21 gene replacing E3gp19k region of the adenovirus genome. An extra sequence ATTTAAAT was left in the virus construct pAd-IL21 after removal of Chloroform (FIG. 18).

### Example 4. Modification of Virus Construct pAd3d-hIL21

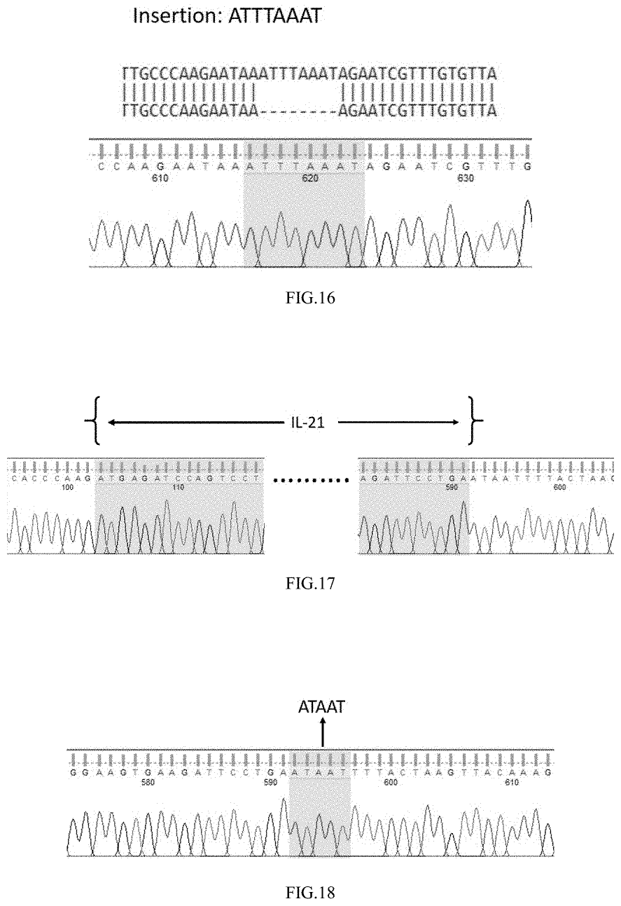

Sequencing result confirms the Y477A mutation, TYAT deletion and A20 insertion (FIG. 16, 17).

An extra sequence ATAAT was left in the virus construct pAd-IL21 after removal of Chloroform (FIG. 15).

### Example 5. hIL-21 Expression in Modified Adenovirus

The expression of hIL-21 was measured by ELISA in the cell culture medium from Ad-hIL-21, Ad-hIL-21-A20 viruses (FIG. 19).

### Example 6. Infection of αvβ6 Integrin Negative or Positive Tumor Cells by Control Virus and A20 Virus

### Example 7 the Modified Virus Ad5 of the Present Application is Capable of Specifically Targeting and Killing a Tumor Cell

The modified virus Ad5 KMAd1 was transfected with several kinds of tumor cells, and the tumor cells without administrated with the virus were regarded as a control.

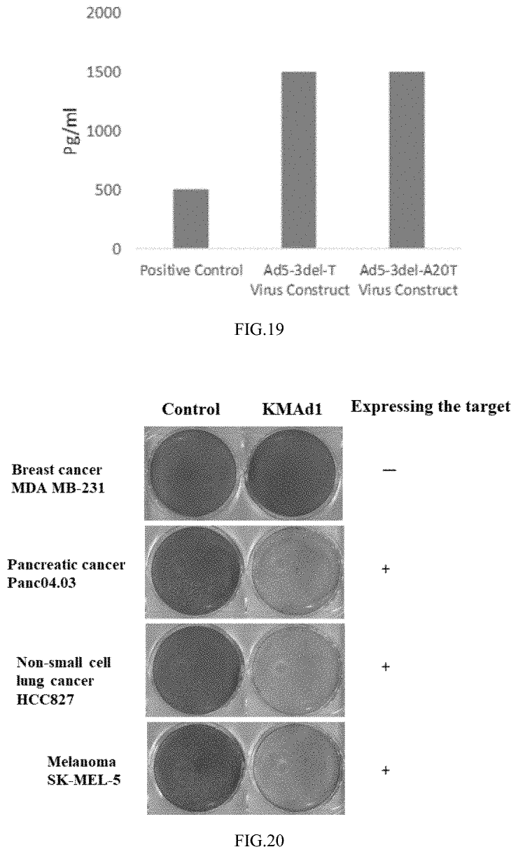

After 3 days of incubation, the cells were stained with crystal violet, and the results are shown in FIG. 20. The results show that modified virus Ad5 of the present application is able to specifically bind and/or kill a αvβ6 integrin positive tumor cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A modified virus Ad5, wherein the modified virus Ad5 is capable of expressing a cytokine, and the modified virus Ad5 is capable of expressing an A20.

2. The modified virus Ad5 of claim 1, wherein the cytokine is originated from human.

3. The modified virus Ad5 of claim 1, wherein the cytokine comprises an interleukin, a tumor necrosis factor, an interferon, a chemokine, a lymphokine and/or a growth factor.

4. The modified virus Ad5 of claim 1, wherein the cytokine comprises an IL12, an IL2, an IL15, an IL8 and/or an IL-21.

5. (canceled)

6. (canceled)

7. The modified virus Ad5 of claim 1, wherein the A20 is originated from a foot-and mouth disease virus (FMDV).

8. The modified virus Ad5 of claim 1, wherein the gene encoding the A20 has a nucleic acid sequence as set forth in SEQ ID NO. 4.

9. The modified virus Ad5 of claim 1, wherein a gene encoding the cytokine and/or the gene encoding the A20 is incorporated in to the genome of the modified virus Ad5.

10. The modified virus Ad5 of claim 1, wherein a gene encoding the A20 is incorporated in to the HI-loop of the modified virus Ad5.

11. The modified virus Ad5 of claim 9, wherein the incorporation uses a method of gene editing and/or gene recombination.

12. The modified virus Ad5 of claim 1, wherein the modified virus Ad5 has at least one modification in fibre region.

13. The modified virus Ad5 of claim 12, wherein the modification in fibre region comprises an amino acid substitution Y477A.

14. The modified virus Ad5 of claim 13, wherein the modification in fibre region comprises a deletion of amino acids TAYT at the residues 489-492.

15. The modified virus Ad5 of claim 1, wherein compared to a wild virus Ad5, the expression and/or activity of a E1ACR2 gene E1B19K gene, and/or a E3gp19K gene is downregulated in the modified virus Ad5.

16. (canceled)

17. (canceled)

18. (canceled)

19. The modified virus Ad5 of claim 15, wherein the downregulation uses a method of gene editing and/or gene recombination.

20. The modified virus Ad5 of claim 19, wherein the gene editing uses an anti-sense RNA, a siRNA, a shRNA and/or a CRISPR/Cas system.

21. The modified virus Ad5 of claim 15, wherein at least a portion of the genes encoding the E1ACR2 gene, at least a portion of the E1B19K gene and/or at least a portion of the E3gp19K is deleted.

22. The modified virus Ad5 of claim 15, wherein the gene encoding a cytokine is incorporated in to the site of the E1ACR2 gene, the E1B19K gene or the E3gp19K gene.

23. The modified virus Ad5 of claim 1, wherein the modified virus Ad5 is capable of expressing a gene and/or a ligand targeting a T cell, a gene and/or a ligand targeting a tumor cell, and/or a therapeutic gene.

24. The modified virus Ad5 of claim 23, wherein the therapeutic gene is selected from a group consisting of: a gene encoding an immune co-stimulatory pathway activating molecule, a gene encoding a checkpoint inhibitor, a gene encoding a cytotoxic, a gene encoding a tumor suppressor gene, and an anti-angiogenesis gene.

25. The modified virus Ad5 of claim 24, wherein the immune co-stimulatory pathway activating molecule is selected from a group consisting of: a CD40 ligand (CD40L), a ICOS ligand, a GITR ligand, a 4-1BB ligand, an OX40 ligand, a TL1A, a CD30 ligand, a CD27, and a Flt3 ligand or the variant thereof.

26. The modified virus Ad5 of claim 24, wherein the checkpoint inhibitor is selected from a group consisting of: a PD-1 inhibitor, a PD-L1 inhibitor and a CTLA-4 inhibitor.

27. The modified virus Ad5 of claim 24, wherein the tumor suppressor gene comprises HIC1 gene.

28. An isolated nucleic acid molecule, encoding the modified virus Ad5 of claim 1.

29. A vector, comprising the isolated nucleic acid molecule of claim 28.

30. A cell, comprising the modified virus Ad5 of claim 1.

31. A pharmaceutical composition, wherein the pharmaceutical composition comprises the modified virus Ad5 of claim 1 and a pharmaceutically accepted adjuvant.

32. A method of treating a disease and/or a disorder, comprising administrating the modified virus Ad5 of claim 1 to a subject in need of.

33. The method of claim 32, comprising administrating the modified virus Ad5 of claim 1 to a subject in need of in a combination of at least one agent, and the agent is selected from a group consisting of an anti-cancer agent, an agonist, an antagonist, a chemotherapeutic agent and a radiation agent.

34. The method of claim 32, wherein the disease comprises a tumor.

35. The method of claim 32, wherein the disease comprises a tumor expressing αvβ6 integrin.

36. The method of claim 32, wherein the disease comprises a pancreatic cancer, a head and neck cancer, and/or an ovarian cancer.

37. (canceled)

38. (canceled)

39. (canceled)

40. (canceled)
